(19) United States
(12) Patent Application Publication
(10) Pub. No.: US 2006/0141627 A1
(43) Pub. Date: Jun. 29, 2006

(54) Vectors for stable gene expression

(75) Inventor: Allen Comer, Madison, WI (US)

Correspondence Address: Medlen & Carroll, LLP, Suite 350, 101 Howard Street, San Francisco, CA, 94105, US

(73) Assignee: Stratatech Corporation, Madison, WI (US)

(21) Appl. No.: 11/282,283

(22) Filed: Nov. 18, 2005

Related U.S. Application Data
(60) Provisional application No. 60/629,148, filed on Nov. 18, 2004.
(60) Provisional application No. 60/632,701, filed on Dec. 2, 2004.

(51) Int. Cl. C12N 15/74

(52) U.S. Cl. 435/472

(57) ABSTRACT

The present invention relates to expression vectors capable of promoting transgene expression. The expression vectors include site specific recombination elements, insulator elements, and recombinase elements. In particular, the present invention provides methods for obtaining specific and stable integration of nucleic acids into eukaryotic cells through site specific recombination.

This application claims priority to U.S. Provisional Application Nos. 60/629,148, filed Nov. 18, 2004, and 60/632,701, filed Dec. 2, 2004. Each of which are incorporated herein by reference.

### FIELD OF THE INVENTION

The present invention relates to expression vectors capable of promoting transgene expression. The expression vectors include site specific recombination elements, insulator elements, and recombinase coding sequences. In particular, the present invention provides methods for obtaining specific and stable integration of nucleic acids into eukaryotic cells through site specific recombination.

### BACKGROUND OF THE INVENTION

Genetic transformation of eukaryotes often suffers from significant shortcomings. For example, it is often difficult to reproducibly obtain integration of a transgene at a particular locus of interest. Homologous recombination generally occurs only at a very low frequency. To overcome this problem, site-specific recombination systems are employed. Site specific recombination generally involves the use of recombinant sites and recombinase proteins.

The Cre-lox system of bacteriophage PI, and the FLP-FRT system of see e,g., Saccharomyces cerevisiae are widely used for transgene and chromosome engineering in animals and plants (see, e.g., Sauer (1994) Curr. Opin. Biotechnol. 5: 521-527; Ow (1996) Curr. Opin. Biotechnol. 7: 181-186). Other systems that operate in animal or plant cells include the following: 1) the R—RS system from Zygosaccharomyces rouxii (see e,g., Onouchi et al. (1995) Mol. Gen. Genet. 247: 653-660), 2) the Gin-gix system from bacteriophage Mu (see e,g., Maeser & Kahmann (1991) Mol. Gen. Genet. 230: 170-176) and, 3) the β-recombinase-six system from bacterial plasmid pSM19035 (see e,g., Diaz et al. (1999) J. Biol. Chem. 274: 6634-6640). By using the site-specific recombinases, one can obtain a greater frequency and specificity of integration.

However, these systems suffer from a significant shortcoming. Each of these systems have in common the property that a single polypeptide recombinase catalyzes the recombination between two sites of identical or nearly identical sequences. The product-sites generated by recombination are themselves substrates for subsequent recombination. Consequently, recombination reactions are readily reversible. Since the kinetics of intramolecular interactions are favored over intermolecular interactions, these recombination systems are efficient for deleting rather than integrating DNA.

An additional problem with the expression of foreign genes in eukaryotic cells is the clonal variation in the expression of the same gene in independent transformants: a problem referred to as “position effect” variation. No completely satisfactory method of obviating this problem has yet been developed.

Thus, a need exists for methods and systems for obtaining stable site-specific integration of genes of interest. Additionally, a need exists for reducing position effect variation.

### SUMMARY OF THE INVENTION

The present invention relates to expression vectors capable of promoting transgene expression. The expression vectors include site specific recombination elements, insulator elements, and recombinase coding sequences. In particular, the present invention provides methods for obtaining specific and stable integration of nucleic acids into eukaryotic cells through site specific recombination.

In certain embodiments, the present invention provides an expression vector comprising a promoter, a transgene, a site specific recombination site, and an insulator element(s). In other embodiments, the expression vector contains a promoter, a site specific recombination site, and an insulator element(s), and a restriction enzyme site for insertion of a transgene of interest. The invention further provides a second expression vector that encodes a recombinase protein capable of catalyzing the integration of the first expression vector into a host cell genome. In some embodiments, the promoter is an epidermal cell specific promoter, while in further embodiments, the promoter is a keratinocyte specific promoter. In preferred embodiments, the promoter is a keratin-5 (K5), involucrin (INV), or keratin-14 (K14) promoter. In other embodiments, the transgene is VEGF. In other embodiments the transgene is KGF-2. In preferred embodiments, the site specific recombination site is attB. In preferred embodiments, the insulator element is HS-4. In further embodiments, the HS-4 is an HS-4 dimer.

In some embodiments, the recombinase element is selected from the group consisting of a bacteriophage φC31 integrase, a coliphage P4 recombinase, a Listeria phage recombinase, a bacteriophage R4 Sre recombinase, a CisA recombinase, an XisF recombinase, and a transposon Tn4451 TnpX recombinase. In preferred embodiments, the recombinase element is a φC31 integrase.

In preferred embodiments, the recombinase coding sequence is operably linked to the promoter in the second expression vector. In preferred embodiments, the transgene is operably linked to the promoter.

In certain embodiments, the present invention provides an expression vector comprising a promoter, a gene of interest, a site specific recombination site, and an insulator element. In preferred embodiments, the promoter is a keratinocyte promoter. In further embodiments, the promoter is K-14. In preferred embodiments, the transgene is a transgene. In further embodiments, the transgene is VEGF. In still further embodiments, the transgene is KGF-2. In preferred embodiments, the site specific recombination site is attB. In preferred embodiments, the insulator element is HS-4. In further embodiments the HS-4 is an HS-4 dimer.

In some preferred embodiments, an additional expression vector comprising a recombinase element is provided. In some embodiments, the recombinase element is selected from the group consisting of a bacteriophage φC31 integrase, a coliphage P4 recombinase, a Listeria phage recombinase, a bacteriophage R4 Sre recombinase, a CisA recombinase, an XisF recombinase, and a transposon Tn4451 TnpX recombinase. In preferred embodiments, the recombinase element is a φC31 integrase.

In preferred embodiments, the recombinase element is operably linked to a promoter. In some embodiments, the promoter is an epidermal cell specific promoter. In further preferred embodiments, the promoter is a keratinocyte specific promoter.

### DESCRIPTION OF THE FIGURES

FIG. 1 provides the consensus sequence of the K14 promoter (SEQ ID NO: 1).

FIG. 2 provides the consensus sequence for the involucrin promoter (SEQ ID NO: 2).

FIG. 3 shows an expression vector of the present invention.

FIG. 4 provides the consensus sequence for VEGF (SEQ ID NO: 3).

FIG. 5 provides a full length HS4 Insulator sequence (SEQ ID NO: 4), the HS4 core sequence (SEQ ID NO: 5), and the HS4 dimer sequence (SEQ ID NO: 6).

FIG. 6 provides an attB recombination site sequence (SEQ ID NO: 7).

FIG. 7 provides a complete vector sequence (SEQ ID NO: 8).

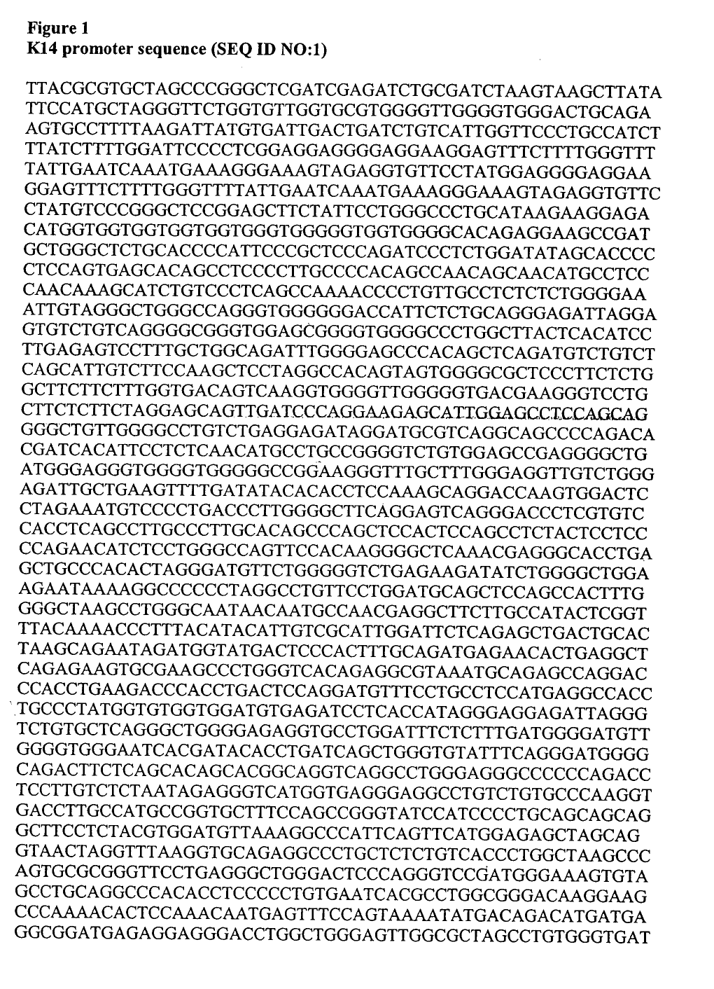

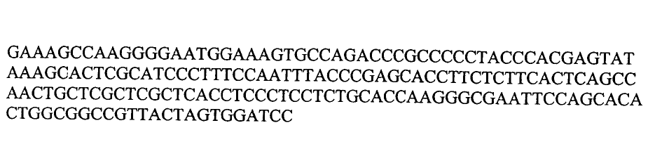

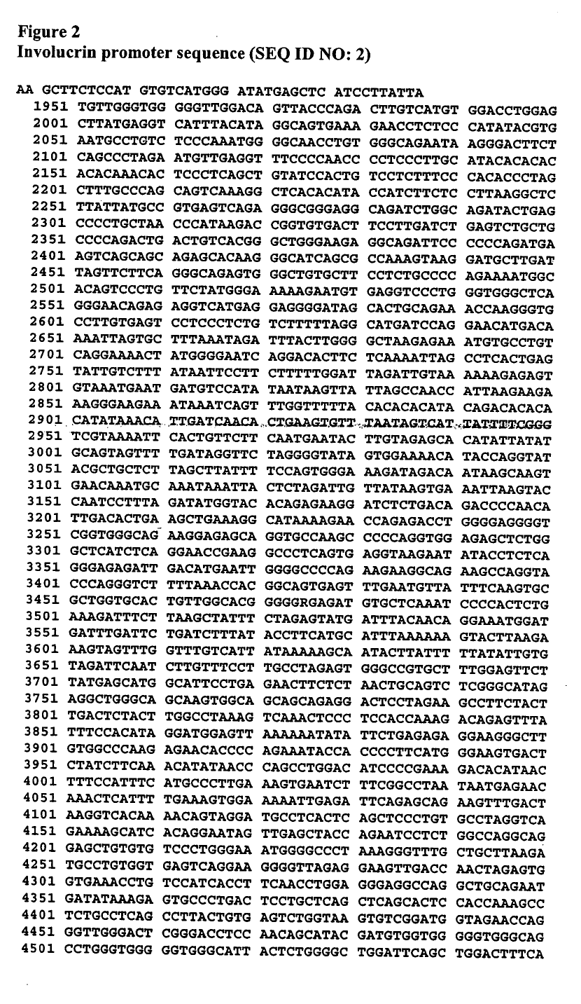

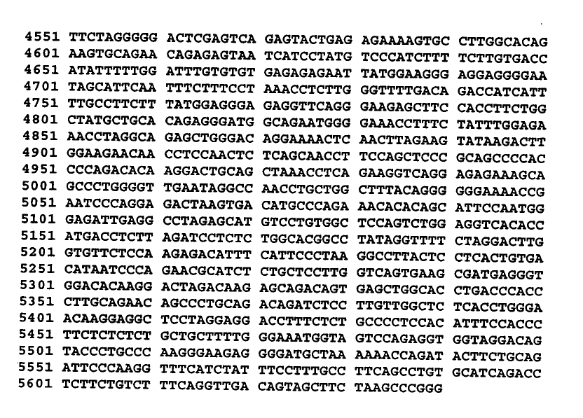

### DEFINITIONS

To facilitate understanding of the invention, a number of terms are defined below.

As used herein, the term “insulator elements,” “insulator borders,” and related terms refer to chromosomal elements capable of hindering the effect of transcriptional enhancers on promoters, and protect the transcription of transgenes from both positive and negative chromosomal position effect variegation. Examples of insulator elements include, but are not limited to, HS2, HS3, and HS4. The terms “HS2”, “HS3” and “HS4” refer to full-length insulator elements as well as elements that are derived from the full length insulator elements such as fragments of the insulator elements (e.g., HS4 fragments as exemplified herein).

As used herein, the term “growth factor” refers to extracellular molecules that bind to a cell-surface triggering an intracellular signaling pathway leading to proliferation, differentiation, or other cellular response. Examples of growth factors include, but are not limited to, growth factor I, trophic factor, Ca²⁺, insulin, hormones, synthetic molecules, pharmaceutical agents, and LDL.

As used herein, the term “keratinocyte growth factor” or “KGF” refers to a member of a group of structurally distinct proteins known as FGFs that display varying degrees of sequence homology, suggesting that they are encoded by a related family of genes. The FGFs share common receptor sites on cell surfaces. KGF, for example, can bind to FGFR-3.

As used herein, the term “NIKS cells” refers to cells having the characteristics of the cells deposited as cell line ATCC CRL-12191.

The term “gene” refers to a nucleic acid (e.g., DNA) sequence that comprises coding sequences necessary for the production of a polypeptide, RNA or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “recombinase” refers to an enzyme that catalyzes recombination between two or more recombination sites. Recombinases useful in the present invention catalyze recombination at specific recombination sites which are specific polynucleotide sequences that are recognized by a particular recombinase. The term “integrase” refers to a type of recombinase.

The terms “recombination elements” and “recombination sites” refer to specific polynucleotide sequences that are recognized by the recombinase enzymes described herein. Typically, two different sites are involved (termed “complementary sites”), one present in the target nucleic acid (e.g., a chromosome or episome of a eukaryote) and another on the nucleic acid that is to be integrated at the target recombination site. The terms “attB,” “attP,” “attL,” and “attR” which refer to attachment (or recombination) sites originally from a bacterial target and a phage donor, respectively, are used herein although recombination sites for particular enzymes may have different names. Recombination elements which share sequence or functional similarity to the bacterial/phage recombination sites are present in mammalian genomes and are also defined as recombination elements herein.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence 5′-“A-G-T-3′,” is complementary to the sequence 3′-“T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Complementarity can include the formation of base pairs between any type of nucleotides, including non-natural bases, modified bases, synthetic bases and the like.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

As used herein the term “portion” when in reference to a protein (as in “a portion of a given protein”) refers to fragments of that protein. The fragments may range in size from four consecutive amino acid residues to the entire amino acid sequence minus one amino acid.

The term “gene of interest” as used herein refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term gene of interest thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis).

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

### DETAILED DESCRIPTION OF THE INVENTION

The present invention provides methods for obtaining site-specific recombination of a gene of interest in eukaryotic cells. The products of the recombinations performed using the methods of the present invention are stable. Thus, one can use the methods to, for example, introduce transgenes into chromosomes of eukaryotic cells and avoid the excision of the transgene that often occurs using previously known site-specific recombination systems. Stable inversions, translocations, and other rearrangements can also be obtained.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is herein incorporated by reference in its entirety.

I. Site Specific Recombination

Many bacteriophage and integrative plasmids encode site-specific recombination systems that enable the stable incorporation of their genome into those of their hosts. In these systems, the minimal requirements for the recombination reaction are a recombinase enzyme, or integrase, which catalyzes the recombination event, and two recombination sites (Sadowski (1986) J. Bacteriol. 165: 341-347; Sadowski (1993) FASEB J. 7: 760-767; each herein incorporated by reference in their entireties). For phage integration systems, these are referred to as attachment (att) sites, with an attP element from phage DNA and the attB element encoded by the bacterial genome. The two attachment sites can share as little sequence identity as a few base pairs. The recombinase protein binds to both att sites and catalyzes a conservative and reciprocal exchange of DNA strands that result in integration of the circular phage or plasmid DNA into host DNA.

The methods of the present invention employ site-specific recombination systems to achieve stable integration or other rearrangement of nucleic acids in eukaryotic cells. Generally, a site-specific recombination system typically consists of three elements: two specific DNA sequences (“the recombination sites”) and a specific enzyme (“the recombinase”) (see, e.g., U.S. Pat. No. 6,746,780; herein incorporated by reference in its entirety). The recombinase catalyzes a recombination reaction between the specific recombination sites. Integration of an expression vector containing one recombination site by recombination with a second recombination site in the genome of a host cell results in the entire integrated expression vector being flanked by two hybrid recombination sites.

Recombination sites have an orientation. The orientation of the recombination sites in relation to each other determines what recombination event takes place. The recombination sites may be in two different orientations: parallel (same direction) or opposite. When the recombination sites are present on a single nucleic acid molecule and are in a parallel orientation to each other, then the recombination event catalyzed by the recombinase is typically an excision of the intervening nucleic acid, leaving a single recombination site. When the recombination sites are in the opposite orientation, then any intervening sequence is typically inverted.

The recombinases used in the methods of the present invention mediate site-specific recombination between a first recombination site and a second recombination site that can serve as a substrate for recombination with the first recombination site. However, in the absence of an additional factor that is not normally present in eukaryotic cells, eukaryotic cells cannot mediate recombination between two hybrid recombination sites that are formed upon recombination between the first recombination site and the second recombination site. Examples of such recombinases include, for example, the bacteriophage φC31 integrase (see, e.g., Thorpe & Smith (1998) Proc. Nat'l. Acad. Sci. USA 95: 5505-5510; Kuhstoss & Rao (1991) J. Mol. Biol. 222: 897-890; U.S. Pat. No. 5,190,871), a phage P4 recombinase (Ow & Ausubel (1983) J. Bacteriol. 155: 704-713), a Listeria phage recombinase, a bacteriophage R4 Sre recombinase (Matsuura et al. (1996) J. Bacteriol. 178: 3374-3376), a CisA recombinase (Sato et al. (1990) J. Bacteriol. 172: 1092-1098; Stragier et al. (1989) Science 243: 507-512), an XisF recombinase (Carrasco et al. (1994) Genes Dev. 8: 74-83), and a transposon Tn4451 TnpX recombinase (Bannam et al. (1995) Mol. Microbiol. 16: 535-551; Crelin & Rood (1997) J. Bacteriol. 179: 5148-5156; each herein incorporated in their entireties).

Recombinase polypeptides, and nucleic acids that encode the recombinase polypeptides, are described in the art and can be obtained using routine methods. For example, a vector that includes a nucleic acid fragment that encodes the φC31 integrase is described in U.S. Pat. No. 5,190,871 (additionally, see, e.g., Andreas, et al., Nucleic Acids Research, 30, 11, 2299-2306 (2002); Ortiz-Urda, et al., Nature Medicine, 8, 10, 1166-1170 (2002); Groth, et al., PNAS, 97(11), 5995-6000 (2000); Olivares, et al., Nature Biotech., 20, 1124-1128 (2002); Thorpe, et al., PNAS 95, 5505-5510 (1998); Baer, et al., Current Opin. Biotech., 12, 473-480 (2001); each herein incorporated by reference in their entireties).

The recombinases can be introduced into the eukaryotic cells that contain the recombination sites at which recombination is desired by any suitable method. For example, one can introduce the recombinase in polypeptide form, e.g., by microinjection or other methods. In presently preferred embodiments, however, a gene that encodes the recombinase is introduced into the cells. Expression of the gene results in production of the recombinase, which then catalyzes recombination among the corresponding recombination sites. One can introduce the recombinase gene into the cell before, after, or simultaneously with, the introduction of the exogenous polynucleotide of interest. In one embodiment, the recombinase gene is present on a separate vector and the vector encoding the recombinase and the vector encoding the gene of interest are cotransfected. In other embodiments, the recombinase gene is introduced into a transgenic eukaryotic organism, e.g., a transgenic plant, animal, fungus, or the like, which is then crossed with an organism that contains the corresponding recombination sites.

In some embodiments, the present invention employs prokaryotic recombinases, such as bacteriophage integrases, that are unidirectional in that they can catalyze recombination between two complementary recombination sites, but cannot catalyze recombination between the hybrid sites that are formed by this recombination. One such recombinase, the φC31 integrase, by itself catalyzes only an attB×attP reaction. The integrase cannot mediate recombination between the attL and attR sites that are formed upon recombination between attB and attP. Because recombinases such as the φC31 integrase cannot alone catalyze the reverse reaction, the φC31 attB×attP recombination is stable. This property is one that sets the methods of the present invention apart from site-specific recombination systems currently in use for eukaryotic cells, such as the Cre-lox or FLP-FRT system, where the recombination reactions can readily reverse. Use of the recombination systems of the invention provides new opportunities for directing stable transgene and chromosome rearrangements in eukaryotic cells.

The methods of the present invention involve contacting a pair of recombination sites (e.g., attB and attP) that are present in a eukaryotic cell with a corresponding recombinase. The recombinase then mediates recombination between the recombination sites. Depending upon the relative locations of the two recombination sites, any one of a number of events can occur as a result of the recombination. For example, if the two recombination sites are present on different nucleic acid molecules, the recombination can result in integration of one nucleic acid molecule into a second molecule. Thus, one can obtain integration of a plasmid that contains one recombination site into a eukaryotic cell chromosome that includes the corresponding recombination site. Because the recombinases used in the methods of the invention cannot catalyze the reverse reaction, the integration is stable. Such methods are useful, for example, for obtaining stable integration into the eukaryotic chromosome of a gene of interest that is present on the plasmid.

As discussed in more detail below, preferred embodiments of the present invention provide vectors containing site-specific recombinases (e.g., ΦC31 integrase) for triggering recombination between a recombination site in the expression vector (e.g., attB) and a different recombination site (e.g., attP site or pseudo-attP site in the case of human cells) into the chromosome of a host cell (e.g., human chromosome 8). Such vectors permit the generation of recombinants prepared by integrating a foreign gene into a host chromosome of a host cell.

II. Insulator Elements

A difficulty encountered with the introduction of transgenic constructs into vertebrate species involves clonal variation in the expression of the same gene in independent transformants. This problem is referred to as “position effect” variation and is thought to relate to the effects of DNA sequences adjacent to the site where the gene was inserted in the genome. In addition to variation of expression, sometimes the expression of the gene product is not regulated in accordance with the promoter used to express the gene. For example, expression of the gene product may occur in non-targeted tissues even though a tissue specific promoter was used to express the gene product. No completely satisfactory method of obviating these problems has yet been developed, and thus there is a continued need for a solution.

Problems relating to the controlled expression of introduced genes arise because the introduced gene may be inserted adjacent to regulatory elements normally present in the genome. For example, it is known that enhancer elements can significantly increase the expression of adjacent genes. Thus if an introduced gene construct was inserted next to a strong enhancer element, the regulatory control of a tissue-specific promoter may be overridden by the enhancer, thus resulting in expression of the gene in non-targeted tissues. Undesired effects on transgene expression are also encountered when transgenes integrate into poorly-expressed regions of the host cell genome (heterochromatin). Accordingly, to increase the predictability and safety of expressing foreign genes in vertebrate species a method must be provided that minimizes these “position effects.” The present invention provides insulators (e.g., HS2, HS3, HS4) that block enhancer activity and other regulatory effects, thus allowing for a more predictable expression pattern for introduced gene constructs. Insulator elements also have the ability to minimize the negative influence of adjacent heterochromatic regions on transgene expression.

Insulators are nucleic acid sequences that function to block enhancer effects on genes, and therefore insulators can be used to block position effects and allow for better regulation of transfected genes. Insulator elements have been described in several nonvertebrate organisms (see, e.g., U.S. Patent Publication Nos. 2003/0211581A1 and 2003/0022303A1, and Geyer, et al., Cell. Mol. Life Sci. (2002) 2112-2127; Taboit-Dameron, et al., Transgenic Research 8 (1999) 223-235; Chung, et al., Cell 74 (1993) 505-514; Szabo, et al., Development 129 (2002) 897, 904; Chung, et al., PNAS 94 (1997) 575-580; Recillas-Targa, et al., PNAS 99 (2002) 6883-6888; Emery, et al., PNAS 97 (2000) 9150-9155). However, the use of insulator elements in conjunction with site specific recombination elements has yet to be described.

As discussed in more detail below, preferred embodiments of the present invention provide cloning vectors containing insulator elements (e.g., HS4) for preventing position effect variation. Other preferred embodiments of the present invention provide expression vectors containing site-specific recombination sites and insulators in a configuration such that, after site-specific integration, the integrated expression vector is flanked by insulator elements.

III. Host Cells

Generally, the present invention is not limited to the use of any particular type of cell or cell line. For example, a number of host cell lines are known in the art and find use in the present invention. In general, these host cells are capable of growth and survival when placed in either monolayer culture or in suspension culture in a medium containing the appropriate nutrients and growth factors, as is described in more detail below. Typically, the cells are capable of expressing and secreting large quantities of a particular protein of interest into the culture medium. Examples of suitable mammalian host cells include, but are not limited to Chinese hamster ovary cells (CHO-K1, ATCC CCl-61); bovine mammary epithelial cells (ATCC CRL 10274; bovine mammary epithelial cells); monkey kidney CV1 line transformed by SV40 (COS-7, ATCC CRL 1651); human embryonic kidney line (293 or 293 cells subcloned for growth in suspension culture; see, e.g., Graham et al., J. Gen Virol., 36:59 [1977]); baby hamster kidney cells (BHK, ATCC CCL 10); mouse sertoli cells (TM4, Mather, Biol. Reprod. 23:243-251 [1980]); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 [1982]); MRC 5 cells; FS4 cells; rat fibroblasts (208F cells); MDBK cells (bovine kidney cells); and a human hepatoma line (Hep G2). The present invention also contemplates the use of amphibian and insect host cell lines. Examples of suitable insect host cell lines include, but are not limited to, mosquito cell lines (e.g., ATCC CRL-1660). Examples of suitable amphibian host cell lines include, but are not limited to, toad cell lines (e.g., ATCC CCL-102).

In some preferred embodiments, the cells are cells from an epidermal cell lineage such as keratinocytes. The present invention is not limited to the use of any particular source of cells that are capable of differentiating into squamous epithelia. Indeed, the present invention contemplates the use of a variety of cell lines and sources that can differentiate into squamous epithelia, including both primary and immortalized keratinocytes. Sources of cells include keratinocytes and dermal fibroblasts biopsied from humans and cavaderic donors (Auger et al., In Vitro Cell. Dev. Biol.—Animal 36:96-103; U.S. Pat. Nos. 5,968,546 and 5,693,332, each of which is incorporated herein by reference), neonatal foreskins (Asbill et al., Pharm. Research 17(9): 1092-97 (2000); Meana et al., Burns 24:621-30 (1998); U.S. Pat. Nos. 4,485,096; 6,039,760; and 5,536,656, each of which is incorporated herein by reference), and immortalized keratinocytes cell lines such as NM1 cells (Baden, In Vitro Cell. Dev. Biol. 23(3):205-213 (1987)), HaCaT cells (Boucamp et al., J. cell. Boil. 106:761-771 (1988)); and NIKS cells (Cell line BC-1-Ep/SL; U.S. Pat. No. 5,989,837, incorporated herein by reference; ATCC CRL-12191).

In particularly preferred embodiments, NIKS cells are utilized. NIKS cells are thoroughly described in U.S. Provisional Patent Application Ser. No. 60/493,664, and U.S. Pat. Nos. 6,514,711, 6,495,135, 6,485,724, 6,214,567, and 5,989,837; each herein incorporated by reference in their entireties. The discovery of a novel human keratinocyte cell line (near-diploid immortalized keratinocytes or NIKS) provides an opportunity to genetically engineer human keratinocytes for new therapeutic methods. A unique advantage of the NIKS cells is that they are a consistent source of genetically-uniform, pathogen-free human keratinocytes. For this reason, they are useful for the application of genetic engineering and genomic gene expression approaches to provide skin equivalent cultures with properties more similar to human skin. Such systems will provide an important alternative to the use of animals for testing compounds and formulations. The NIKS keratinocyte cell line, identified and characterized at the University of Wisconsin, is nontumorigenic, exhibits a stable karyotype, and undergoes normal differentiation both in monolayer and organotypic culture. NIKS cells form fully stratified skin equivalents in culture. These cultures are indistinguishable by all criteria tested thus far from organotypic cultures formed from primary human keratinocytes. Unlike primary cells however, the immortalized NIKS cells will continue to proliferate in monolayer culture indefinitely. This provides an opportunity to genetically manipulate the cells and isolate new clones of cells with new useful properties (Allen-Hoffmann et al., J. Invest. Dermatol., 114(3): 444-455 (2000)).

The NIKS cells arose from the BC-1-Ep strain of human neonatal foreskin keratinocytes isolated from an apparently normal male infant. In early passages, the BC-1-Ep cells exhibited no morphological or growth characteristics that were atypical for cultured normal human keratinocytes. Cultivated BC-1-Ep cells exhibited stratification as well as features of programmed cell death. To determine replicative lifespan, the BC-1-Ep cells were serially cultivated to senescence in standard keratinocyte growth medium at a density of 3×10⁵ cells per 100-mm dish and passaged at weekly intervals (approximately a 1:25 split). By passage 15, most keratinocytes in the population appeared senescent as judged by the presence of numerous abortive colonies which exhibited large, flat cells. However, at passage 16, keratinocytes exhibiting a small cell size were evident. By passage 17, only the small-sized keratinocytes were present in the culture and no large, senescent keratinocytes were evident. The resulting population of small keratinocytes that survived this putative crisis period appeared morphologically uniform and produced colonies of keratinocytes exhibiting typical keratinocyte characteristics including cell-cell adhesion and apparent squame production. The keratinocytes that survived senescence were serially cultivated at a density of 3×10⁵ cells per 100-mm dish. Typically the cultures reached a cell density of approximately 8×10⁶ cells within 7 days. This stable rate of cell growth was maintained through at least 59 passages, demonstrating that the cells had achieved immortality. The keratinocytes that emerged from the original senescencing population were originally designated BC-1-Ep/Spontaneous Line and are now termed NIKS. The NIKS cell line has been screened for the presence of proviral DNA sequences for HIV-1, HIV-2, EBV, CMV, HTLV-1, HTLV-2, HBV, HCV, B-19 parvovirus, HPV-16 and HPV-31 using either PCR or Southern analysis. None of these viruses were detected.

Chromosomal analysis was performed on the parental BC-1-Ep cells at passage 3 and NIKS cells at passages 31 and 54. The parental BC-1-Ep cells have a normal chromosomal complement of 46, XY. At passage 31, all NIKS cells contained 47 chromosomes with an extra isochromosome of the long arm of chromosome 8. No other gross chromosomal abnormalities or marker chromosomes were detected. At passage 54, all cells contained the isochromosome 8.

The DNA fingerprints for the NIKS cell line and the BC-1-Ep keratinocytes are identical at all twelve loci analyzed demonstrating that the NIKS cells arose from the parental BC-1-Ep population. The odds of the NIKS cell line having the parental BC-1-Ep DNA fingerprint by random chance is 4×10⁻¹⁶. The DNA fingerprints from three different sources of human keratinocytes, ED-1-Ep, SCC4 and SCC13y are different from the BC-1-Ep pattern. This data also shows that keratinocytes isolated from other humans, ED-1-Ep, SCC4, and SCC13y, are unrelated to the BC-1-Ep cells or each other. The NIKS DNA fingerprint data provides an unequivocal way to identify the NIKS cell line.

Loss of p53 function is associated with an enhanced proliferative potential and increased frequency of immortality in cultured cells. The sequence of p53 in the NIKS cells is identical to published p53 sequences (GenBank accession number: M14695). In humans, p53 exists in two predominant polymorphic forms distinguished by the amino acid at codon 72. Both alleles of p53 in the NIKS cells are wild-type and have the sequence CGC at codon 72, which codes for an arginine. The other common form of p53 has a proline at this position. The entire sequence of p53 in the NIKS cells is identical to the BC-1-Ep progenitor cells. Rb was also found to be wild-type in NIKS cells.

Anchorage-independent growth is highly correlated to tumorigenicity in vivo. For this reason, the anchorage-independent growth characteristics of NIKS cells in agar or methylcellulose-containing medium was investigated. After 4 weeks in either agar- or methylcellulose-containing medium, NIKS cells remained as single cells. The assays were continued for a total of 8 weeks to detect slow growing variants of the NIKS cells. None were observed.

To determine the tumorigenicity of the parental BC-1-Ep keratinocytes and the immortal NIKS keratinocyte cell line, cells were injected into the flanks of athymic nude mice. The human squamous cell carcinoma cell line, SCC4, was used as a positive control for tumor production in these animals. The injection of samples was designed such that animals received SCC4 cells in one flank and either the parental BC-1-Ep keratinocytes or the NIKS cells in the opposite flank. This injection strategy eliminated animal to animal variation in tumor production and confirmed that the mice would support vigorous growth of tumorigenic cells. Neither the parental BC-1-Ep keratinocytes (passage 6) nor the NIKS keratinocytes (passage 35) produced tumors in athymic nude mice.

NIKS cells were analyzed for the ability to undergo differentiation in both surface culture and organotypic culture. For cells in surface culture, a marker of squamous differentiation, the formation comified envelopes was monitored. In cultured human keratinocytes, early stages of comified envelope assembly result in the formation of an immature structure composed of involucrin, cystatin-α and other proteins, which represent the innermost third of the mature comified envelope. Less than 2% of the keratinocytes from the adherent BC-1-Ep cells or the NIKS cell line produce cornified envelopes. This finding is consistent with previous studies demonstrating that actively growing, subconfluent keratinocytes produce less than 5% cornified envelopes. To determine whether the NIKS cell line is capable of producing comified envelopes when induced to differentiate, the cells were removed from surface culture and suspended for 24 hours in medium made semi-solid with methylcellulose. Many aspects of terminal differentiation, including differential expression of keratins and comified envelope formation can be triggered in vitro by loss of keratinocyte cell-cell and cell-substratum adhesion. The NIKS keratinocytes produced as many as and usually more comified envelopes than the parental keratinocytes. These findings demonstrate that the NIKS keratinocytes are not defective in their ability to initiate the formation of this cell type-specific differentiation structure.

To confirm that the NIKS keratinocytes can undergo squamous differentiation, the cells were cultivated in organotypic culture. Keratinocyte cultures grown on plastic substrata and submerged in medium replicate but exhibit limited differentiation. Specifically, human keratinocytes become confluent and undergo limited stratification producing a sheet consisting of 3 or more layers of keratinocytes. By light and electron microscopy there are striking differences between the architecture of the multilayered sheets formed in tissue culture and intact human skin. In contrast, organotypic culturing techniques allow for keratinocyte growth and differentiation under in vivo-like conditions. Specifically, the cells adhere to a physiological substratum consisting of dermal fibroblasts embedded within a fibrillar collagen base. The organotypic culture is maintained at the air-medium interface. In this way, cells in the upper sheets are air-exposed while the proliferating basal cells remain closest to the gradient of nutrients provided by diffusion through the collagen gel. Under these conditions, correct tissue architecture is formed. Several characteristics of a normal differentiating epidermis are evident. In both the parental cells and the NIKS cell line a single layer of cuboidal basal cells rests at the junction of the epidermis and the dermal equivalent. The rounded morphology and high nuclear to cytoplasmic ratio is indicative of an actively dividing population of keratinocytes. In normal human epidermis, as the basal cells divide they give rise to daughter cells that migrate upwards into the differentiating layers of the tissue. The daughter cells increase in size and become flattened and squamous. Eventually these cells enucleate and form comified, keratinized structures. This normal differentiation process is evident in the upper layers of both the parental cells and the NIKS cells. The appearance of flattened squamous cells is evident in the upper layers of keratinocytes and demonstrates that stratification has occurred in the organotypic cultures. In the uppermost part of the organotypic cultures the enucleated squames peel off the top of the culture. To date, no histological differences in differentiation at the light microscope level between the parental keratinocytes and the NIKS keratinocyte cell line grown in organotypic culture have been observed

To observe more detailed characteristics of the parental (passage 5) and NIKS (passage 38) organotypic cultures and to confirm the histological observations, samples were analyzed using electron microscopy. Parental cells and the immortalized human keratinocyte cell line, NIKS, were harvested after 15 days in organotypic culture and sectioned perpendicular to the basal layer to show the extent of stratification. Both the parental cells and the NIKS cell line undergo extensive stratification in organotypic culture and form structures that are characteristic of normal human epidermis. Abundant desmosomes are formed in organotypic cultures of parental cells and the NIKS cell line. The formation of a basal lamina and associated hemidesmosomes in the basal keratinocyte layers of both the parental cells and the cell line was also noted.

Hemidesmosomes are specialized structures that increase adhesion of the keratinocytes to the basal lamina and help maintain the integrity and strength of the tissue. The presence of these structures was especially evident in areas where the parental cells or the NIKS cells had attached directly to the porous support. These findings are consistent with earlier ultrastructural findings using human foreskin keratinocytes cultured on a fibroblast-containing porous support. Analysis at both the light and electron microscopic levels demonstrate that the NIKS cell line in organotypic culture can stratify, differentiate, and form structures such as desmosomes, basal lamina, and hemidesmosomes found in normal human epidermis.

The present invention contemplates methods and compositions for making cells (e.g. NIKS cells) that express an antimicrobial polypeptide. NIKS cell transformation procedures suitable for use herein are those known in the art and include, for example with mammalian cell systems, dextran-mediated transfection, calcium phosphate precipitation, polybrene-mediated transfection, protoplast fusion, electroporation, encapsulation of the antimicrobial polypeptide polynucleotide in liposomes, and direct microinjection of the DNA into nuclei.

IV. Promoters

In preferred embodiments, the expression vectors of the present invention comprise a promoter which can be operably linked to a gene of interest. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses.

In other embodiments, any promoter that would allow expression of the gene of interest in an epidermal cell host can be used in the present invention. Examples of promoters useful in the present invention include, but are not limited to, K14, K5, and Involucrin promoters.

In preferred embodiments, the human involucrin promoter is used to drive expression of the gene of interest. In such embodiments, a gene of interest is operably linked to the involucrin promoter and transfected into epidermal host cells (e.g., NIKS cells) in an expression vector.

In other preferred embodiments, the K14 promoter is used to drive expression of a gene of interest. In such embodiments, a gene of interest is operably linked to the K14 promoter and transfected into epidermal host cells (e.g., NIKS cells) in an expression vector. In some embodiments, the K14 promoter is isolated from a DNA source, cloned, sequenced, and shuttled into a selection vector. In further embodiments, isolation of the K14 promoter DNA occurs via PCR with K14 primer sequences. Primer sequences specific for K14 Promoter can be obtained from Genbank. Amplification of a DNA source with such primer sequences through standard PCR procedures results in the isolation of K14 Promoter DNA.

A wide variety of genes of interest may be linked to the promoter. Such genes include, but are not limited to, those encoding KGF-2, Defensins 1, 2 or 3, Cathelocidins, VEGF, HIF1α, Ots A and Ots B (see, e.g., International Patent Application No. PCT/US02/06088).

Optionally, other regulatory sequences can be used herein, such as one or more of an enhancer sequence, an intron with functional splice donor and acceptance sites, a signal sequence for directing secretion of the gene of interest, a polyadenylation sequence, other transcription terminator sequences, and a sequence homologous to the host cell genome.

Further, a selectable marker can be present in the expression vector for selection of the presence thereof in the transformed host cells. Selectable markers are genes that encode an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene, which is used in conjunction with CAD-deficient cells, and the mammalian hypoxanthine-guanine phosphoribosyl transferase (hprt) gene, which is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

V. Constructs for Introduction of Exogenous DNA into Target Cells

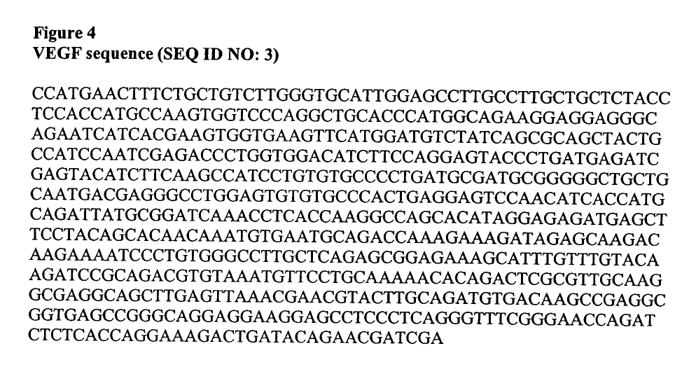

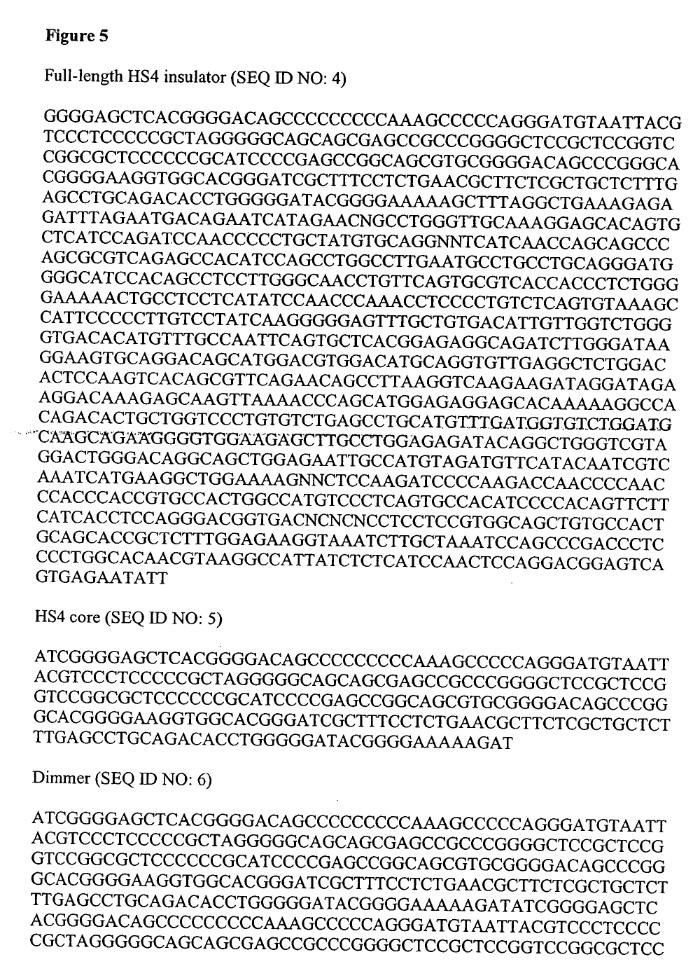

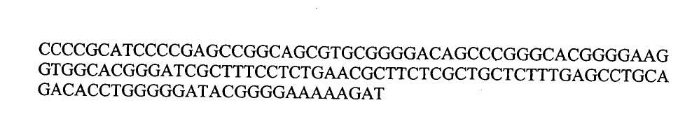

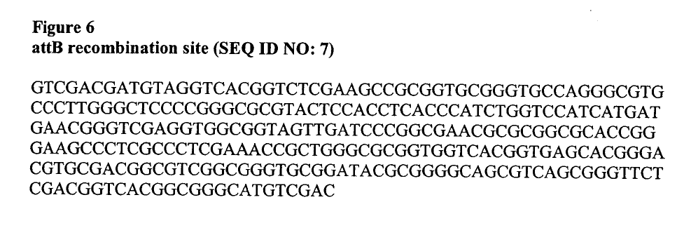

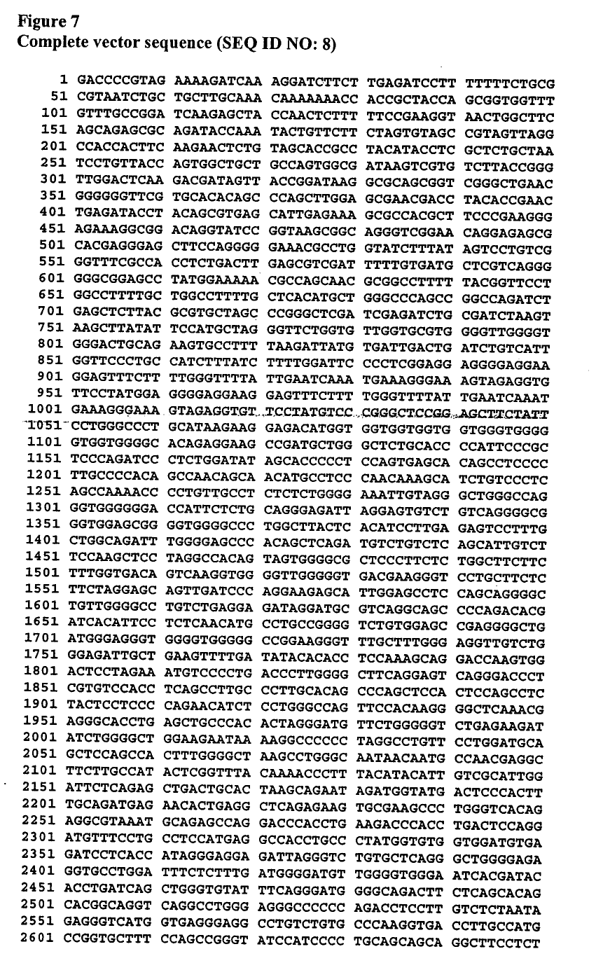

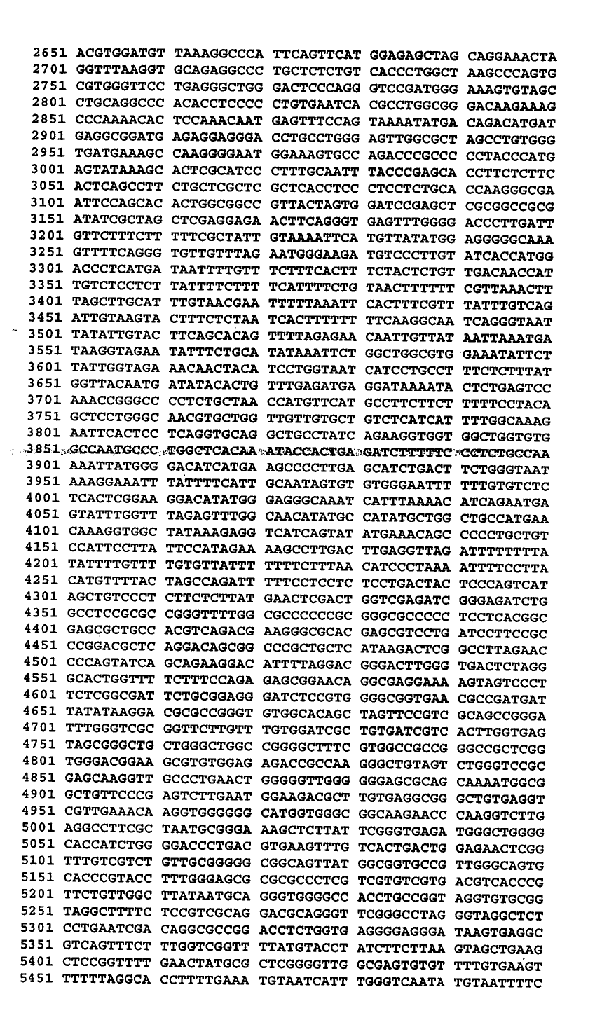

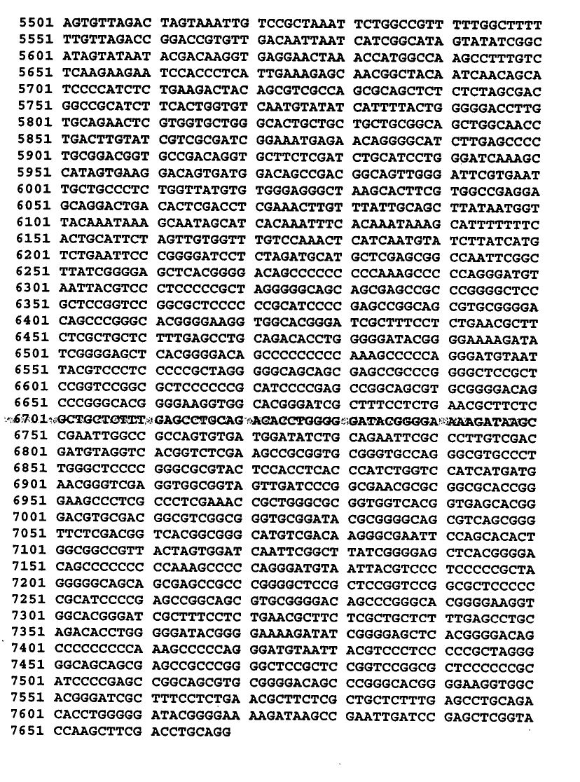

The present invention contemplates cells expressing a gene of interest (e.g., KGF-2, VEGF) and compositions and methods for making cells expressing a gene of interest. The consensus sequence for VEGF is provided at FIG. 4. The present invention is not limited to a particular gene of interest. In preferred embodiments, cells are induced to express a gene of interest through transfection with an expression vector containing the gene of interest DNA. An expression vector containing the gene of interest DNA can be produced by operably linking the gene of interest DNA to one or more regulatory sequences (e.g., K14 promoter) such that the resulting vector is operable in a desired host (e.g., NIKS cells). In preferred embodiments, the regulatory sequence is an epidermal cell promoter regulatory sequence (e.g., K-14, K-5, K-6). The present invention is not limited to a particular promoter.

In certain embodiments, the present invention accomplishes transgene expression in eukaryotic cells through site-specific recombination. As such, the expression vectors are provided containing nucleic acid comprising site-specific recombination elements (e.g., attB), insulator elements (e.g., HS4), promoter sequences (e.g., K14) and a gene of interest sequence (e.g., KGF-2). In preferred embodiments, the components within an expression vector are provided in the following 5′ to 3′ arrangement: recombination element (e.g., attB), insulator element (e.g., HS4), epidermal cell-specific promoter sequence (e.g., K14), gene of interest sequence (e.g., KGF-2), insulator element.

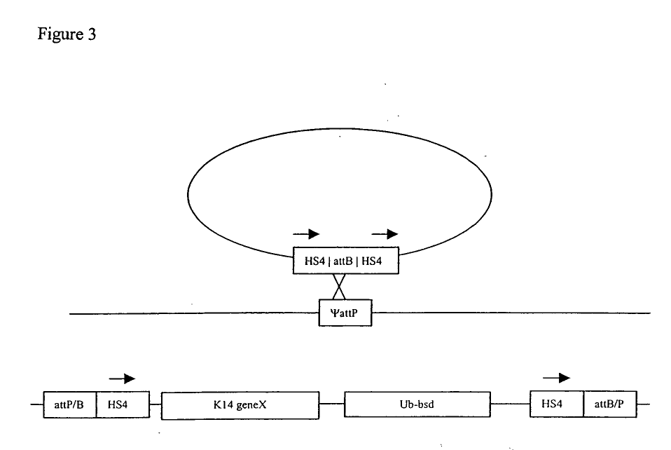

In preferred embodiments, transfection of such expression vectors into eukaryotic cells (e.g., NIKS cells) results in the introduction of the gene of interest into chromosomes of eukaryotic cells and avoids the excision of the transgene that often occurs using previously known site-specific recombination systems. FIG. 3 presents an expression vector with an attB site specific recombination site, HS4 insulator elements and a K14 promoter. In further preferred embodiments, a second expression vector comprising a promoter operably linked to a recombinase sequence (e.g., φC-31 integrase) is co-transfected with the initial expression vector.

### EXAMPLES

### Example 1

### Isolation of HS4 Insulator Element, attB Integration Site, and C31 Integrase

A DNA fragment containing the 250 bp “core” of the HS4 insulator element was amplified by PCR from chicken genomic DNA using primers designed to published HS4 sequences (see, e.g., Chung, J. H., et al., Proc Natl Acad Sci USA, 1997, 94(2): 575-80). This DNA fragment was cloned into the pCR2.1 vector and sequenced to verify its identity and integrity. The HS4 core element was identical to previously published sequences. The HS4 core element was excised from the pCR2.1 vector with EcoRV and multimerized by ligating the 250 bp HS4 monomer overnight and gel-purifying and cloning 500 bp DNA fragments corresponding to HS4 dimers. Plasmids containing HS4 dimers in directly repeated orientation were identified by restriction analysis and DNA sequencing. Dimerized HS4 core elements were termed 2XHS4.

A 285 bp DNA fragment containing the attB integration target sequence was isolated from S. lividans genomic DNA by PCR using primers to published attB sequences (see, e.g., Rausch, H. and M. Lehmann, Nucleic Acids Res, 1991, 19(19): 5187-9). A minimal attB element was also assembled by annealing complementary oligonucleotides to generate a 53 bp double stranded DNA product that contains the minimal attB element (see, e.g., Groth, A. C., et al., Proc Natl Acad Sci USA, 2000, 97(11): 5995-6000). These DNA fragments were cloned into the pCR2.1 vector and sequenced to verify their identity and integrity.

A DNA fragment containing the coding region for C31 integrase was amplified by PCR from the phage ΦC31. The primers used for amplification were designed such that the C31 integrase coding region was preceded by a Kozak consensus translation initiation site and a nuclear localization sequence was introduced immediately downstream of the C31 integrase coding region. This DNA fragment was cloned into the pCR2.1 vector and sequenced to verify its identity and integrity. The C31 integrase coding region was then cloned into an expression vector such that C31 integrase expression is driven by the human K14 promoter.

### Example 2

### Assembly of Insulator/Targeted Integration Cassettes

Cassettes containing the attB integration element flanked by dimerized HS4 elements were assembled in a two-step cloning strategy. First, the 2XHS4 element was cloned into the NotI site of pCR2.1 vectors containing either the 53 bp minimal attB element or the 285 bp attB element described in Example 1. After screening for insert orientation by restriction analysis and DNA sequencing, a second copy of the 2XHS4 element was cloned into the BamHI site of plasmids containing one copy of the 2XHS4 element and either the 53 bp or 285 bp attB elements. Clones that contained copies of the 2XHS4 element in direct orientation were identified by restriction analysis and confirmed by DNA sequencing.

The insulator/attB cassettes were cloned into expression vectors containing the coding region for VEGF₁₆₅.

### Example 3

### Isolation of Stably-Transfected Cells

Stable cell clones with randomly-integrated expression constructs were isolated by transfecting NIKS keratinocytes with a K14-VEGF expression vector lacking the insulator/attB cassette. Stable cell clones that integrated via the attB element were isolated by co-transfecting NIKS keratinocytes with a K14-VEGF expression vector containing the insulator/attB cassette and an expression vector containing the C31 integrase under control of the K14 promoter. In both cases, transfected cells were grown in the presence of blasticidin for three weeks to select for clones that had stably incorporated the K14-VEGF expression constructs. Independent clones were isolated, expanded, and stored in liquid nitrogen as glycerol stocks.

### Example 4

### VEGF Expression in Stably-Transfected Clones

Independent clones containing or lacking the insulator/targeted integration cassette were grown in monolayer culture and also in organotypic culture to produce skin tissue. Conditioned medium from cells or tissue was collected and analyzed by ELISA to quantify VEGF content. On average, the level of VEGF secreted from cells or tissue prepared from clones with the insulator/targeted integration cassette was 6-fold higher than that from clones lacking insulators and isolated by random integration. The data are presented in Table I.

TABLE I
Increased Transgene Expression from Constructs Containing
Insulators and Targeted-integration Sequences

| | | VEGF expression | |
|---|---|---|---|
| Clone | Construct | (fold over endogenous | Average |
| 43:13 4D1 | K14-VEGF | 1.6× | |
| 33:65 A1B | K14-VEGF | 1.5× | |
| 33:12 C9B | K14-VEGF | 1.3× | 1.5× |
| 45:39B | K14-VEGF/attB-HS4 | 10× | |
| 45:53 C4B | K14-VEGF/attB-HS4 | 4× | |
| 55:54 A1 | K14-VEGF/attB-HS4 | 6.5× | 6.8× |

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. An expression vector comprising one or more promoters operably linked to one or more genes of interest and a site-specific recombination element flanked by at least one insulator element on both the 5′ and 3′ sides of said recombination element, wherein upon recombination with a chromosome said expression vector produces an insert with the following 5′ to 3′ arrangement: site-specific recombination remnant, insulator element, genes of interest operably linked to promoters, insulator element, and site-specific recombination recombination remnant.

2. The expression vector of claim 1, wherein said promoter is an epidermal cell specific promoter.

3. The expression vector of claim 1, wherein said epidermal-specific promoter is selected from the group consisting of the K14 promoter and the involucrin promoter.

4. The expression vector of claim 1, wherein said gene of interest is selected from the group consisting of VEGF and KGF-2.

5. The expression vector of claim 1, wherein said site specific recombination element is selected from the group consisting of attB, attP, attL, and attR.

6. The expression vector of claim 1, wherein said insulator element is HS-4.

7. The expression vector of claim 6, wherein said HS-4 is a HS-4 dimer.

8. A system for introducing a gene into the genome of a host cell comprising: 1) the expression vector of claim 1; and 2) an expression vector comprising a promoter operably linked to a recombinase.

9. The system of claim 8, wherein said recombinase is selected from the group consisting of a bacteriophage φC31 integrase, a coliphage P4 recombinase, a Listeria phage recombinase, a bacteriophage R4 Sre recombinase, a CisA recombinase, an XisF recombinase, and a transposon Tn4451 TnpX recombinase.

10. The system of claim 8, wherein said recombinase is a φC31 integrase.

11. A kit for generating a recombinant expression vector for integrating a gene of interest DNA sequence, comprising:
a) the expression vector of claim 1;
b) a second expression vector comprising a promoter operably linked to a recombinase; and
c) instructions for integrating a gene of interest in cells.

12. The kit of claim 11, wherein said promoter sequence is selected from the group consisting of K14 and involucrin.

13. The kit of claim 11, wherein said at least one site-specific recombination site is selected from the group consisting of attB, attP, attL, and attR.

14. The kit of claim 11, wherein said at least one insulator group is HS4.

15. The expression vector of claim 14, wherein said HS-4 is a HS-4 dimer.

16. The kit of claim 11, wherein said recombinase is selected from the group consisting of a bacteriophage φC31 integrase, a coliphage P4 recombinase, a Listeria phage recombinase, a bacteriophage R4 Sre recombinase, a CisA recombinase, an XisF recombinase, and a transposon Tn4451 TnpX recombinase.

17. The kit of claim 11, wherein said recombinase is a φC31 integrase.

18. A method of expressing a gene of interest in a host cell, comprising:
a) providing:
i) a first expression vector comprising a promoter operably linked to said gene of interest and a site-specific recombination element flanked by at least one insulator element on both the 5′ and 3′ sides of said recombination element;
ii) a second expression vector comprising a promoter operably linked to a recombinase; and
iii) host cells;
b) introducing said first and second expression vectors into said host cells under conditions such that recombinase protein produced from said second expression vector causes integration of said first expression vector via said recombination element.

19. The method of claim 18, wherein said promoter is selected from the group consisting of the K14 promoter and the involucrin promoter.

20. The method of claim 18, wherein said gene of interest is selected from the group consisting of VEGF and KGF-2.

21. The method of claim 18, wherein said site specific recombination element is selected from the group consisting of attB, attP, attL, and attR.

22. The method of claim 18, wherein said insulator element is HS-4.

23. The method of claim 22, wherein said HS-4 is a HS-4 dimer.

24. The method of claim 18, wherein said recombinase is selected from the group consisting of a bacteriophage φC31 integrase, a coliphage P4 recombinase, a Listeria phage recombinase, a bacteriophage R4 Sre recombinase, a CisA recombinase, an XisF recombinase, and a transpo son Tn4451 TnpX recombinase.

25. The method of claim 18, wherein said recombinase is a φC31 integrase.

26. The method of claim 18, wherein said host cell is an epidermal host cell.

27. The method of claim 26, wherein said epidermal host cell is a keratinocyte.


SEQUENCE LISTING (8 sequences)

SEQ ID NO: 1 - DNA, 2429 bp, Homo sapiens
ttacgcgtgctagcccgggctcgatcgagatctgcgatctaagtaagcttatattccatgctagggttctggtgttggtgcgtggggttggggtgggactgcagaagtgccttttaagattatgtgattgactgatctgtcattggttccctgccatctttatcttttggattcccctcggaggaggggaggaaggagtttcttttgggttttattgaatcaaatgaaagggaaagtagaggtgttcctatggaggggaggaaggagtttcttttgggttttattgaatcaaatgaaagggaaagtagaggtgttcctatgtcccgggctccggagcttctattcctgggccctgcataagaaggagacatggtggtggtggtggtgggtgggggtggtggggcacagaggaagccgatgctgggctctgcaccccattcccgctcccagatccctctggatatagcaccccctccagtgagcacagcctccccttgccccacagccaacagcaacatgcctcccaacaaagcatctgtccctcagccaaaacccctgttgcctctctctggggaaattgtagggctgggccagggtggggggaccattctctgcagggagattaggagtgtctgtcaggggcgggtggagcggggtggggccctggcttactcacatccttgagagtcctttgctggcagatttggggagcccacagctcagatgtctgtctcagcattgtcttccaagctcctaggccacagtagtggggcgctcccttctctggcttcttctttggtgacagtcaaggtggggttgggggtgacgaagggtcctgcttctcttctaggagcagttgatcccaggaagagcattggagcctccagcaggggctgttggggcctgtctgaggagataggatgcgtcaggcagccccagacacgatcacattcctctcaacatgcctgccggggtctgtggagccgaggggctgatgggagggtggggtgggggccggaagggtttgctttgggaggttgtctgggagattgctgaagttttgatatacacacctccaaagcaggaccaagtggactcctagaaatgtcccctgacccttggggcttcaggagtcagggaccctcgtgtccacctcagccttgcccttgcacagcccagctccactccagcctctactcctccccagaacatctcctgggccagttccacaaggggctcaaacgagggcacctgagctgcccacactagggatgttctgggggtctgagaagatatctggggctggaagaataaaaggcccccctaggcctgttcctggatgcagctccagccactttggggctaagcctgggcaataacaatgccaacgaggcttcttgccatactcggtttacaaaaccctttacatacattgtcgcattggattctcagagctgactgcactaagcagaatagatggtatgactcccactttgcagatgagaacactgaggctcagagaagtgcgaagccctgggtcacagaggcgtaaatgcagagccaggacccacctgaagacccacctgactccaggatgtttcctgcctccatgaggccacctgccctatggtgtggtggatgtgagatcctcaccatagggaggagattagggtctgtgctcagggctggggagaggtgcctggatttctctttgatggggatgttggggtgggaatcacgatacacctgatcagctgggtgtatttcagggatggggcagacttctcagcacagcacggcaggtcaggcctgggagggccccccagacctccttgtctctaatagagggtcatggtgagggaggcctgtctgtgcccaaggtgaccttgccatgccggtgctttccagccgggtatccatcccctgcagcagcaggcttcctctacgtggatgttaaaggcccattcagttcatggagagctagcaggtaactaggtttaaggtgcagaggccctgctctctgtcaccctggctaagcccagtgcgcgggttcctgagggctgggactcccagggtccgatgggaaagtgtagcctgcaggcccacacctccccctgtgaatcacgcctggcgggacaaggaagcccaaaacactccaaacaatgagtttccagtaaaatatgacagacatgatgaggcggatgagaggagggacctggctgggagttggcgctagcctgtgggtgatgaaagccaaggggaatggaaagtgccagacccgccccctacccacgagtataaagcactcgcatccctttccaatttacccgagcaccttctcttcactcagccaactgctcgctcgctcacctccctcctctgcaccaagggcgaattccagcacactggcggccgttactagtggatcc

SEQ ID NO: 2 - DNA, 3732 bp, Homo sapiens
aagcttctccatgtgtcatgggatatgagctcatccttattatgttgggtgggggttggacagttacccagacttgtcatgtggacctggagcttatgaggtcatttacataggcagtgaaagaacctctcccatatacgtgaatgcctgtctcccaaatggggcaacctgtgggcagaataagggacttctcagccctagaatgttgaggtttccccaacccctcccttgcatacacacacacacaaacactccctcagctgtatccactgtcctctttcccacaccctagctttgcccagcagtcaaaggctcacacataccatcttctccttaaggctcttattatgccgtgagtcagagggcgggaggcagatctggcagatactgagcccctgctaacccataagaccggtgtgacttccttgatctgagtctgctgccccagactgactgtcacgggctgggaagaggcagattccccccagatgaagtcagcagcagagcacaagggcatcagcgccaaagtaaggatgcttgattagttcttcagggcagagtgggctgtgcttcctctgccccagaaaatggcacagtccctgttctatgggaaaaagaatgtgaggtccctgggtgggctcagggaacagagaggtcatgaggaggggatagcactgcagaaaccaagggtgccttgtgagtcctccctctgtctttttaggcatgatccaggaacatgacaaaattagtgctttaaatagatttacttggggctaagagaaatgtgcctgtcaggaaaactatggggaatcaggacacttctcaaaattagcctcactgagtattgtctttataattccttctttttggattagattgtaaaaaagagagtgtaaatgaatgatgtccatataataagttattagccaaccattaagaagaaagggaagaaataaatcagtttggtttttacacacacatacagacacacacatataaacattgatcaacactgaagtgtttaatagtcattattttcgggtcgtaaaattcactgttcttcaatgaatacttgtagagcacatattatatgcagtagttttgataggttctaggggtatagtggaaaacataccaggtatacgctgctcttagcttattttccagtgggaaagatagacaataagcaagtgaacaaatgcaaataaattactctagattgttataagtgaaattaagtaccaatcctttagatatggtacacagagaaggatctctgacagaccccaacattgacactgaagctgaaaggcataaaagaaccagagacctggggaggggtcggtgggcagaaggagagcaggtgccaagcccccaggtggagagctctgggctcatctcaggaaccgaaggccctcagtgaggtaagaatatacctctcagggagagattgacatgaattggggccccagaagaaggcagaagccaggtacccagggtcttttaaaccacggcagtgagtttgaatgttatttcaagtgcgctggtgcactgttggcacgggggrgagatgtgctcaaatccccactctgaaagatttcttaagctatttctagagtatgatttacaacaggaaatggatgatttgattctgatctttataccttcatgcatttaaaaaagtacttaagaaagtagtttggtttgtcattataaaaagcaatacttatttttatattgtgtagattcaatcttgtttccttgcctagagtgggccgtgctttggagttcttatgagcatggcattcctgagaacttctctaactgcagtctcgggcatagaggctgggcagcaagtggcagcagcagaggactcctagaagccttctacttgactctacttggcctaaagtcaaactccctccaccaaagacagagtttatttccacataggatggagttaaaaaatatattctgagagaggaagggcttgtggcccaagagaacaccccagaaataccaccccttcatgggaagtgactctatcttcaaacatataacccagcctggacatccccgaaagacacataactttccatttcatgcccttgaaagtgaatctttcggcctaataatgagaacaaactcattttgaaagtggaaaaattgagattcagagcagaagtttgactaaggtcacaaaacagtaggatgcctcactcagctccctgtgcctaggtcagaaaagcatcacaggaatagttgagctaccagaatcctctggccaggcaggagctgtgtgtccctgggaaatggggccctaaagggtttgctgcttaagatgcctgtggtgagtcaggaaggggttagaggaagttgaccaactagagtggtgaaacctgtccatcaccttcaacctggagggaggccaggctgcagaatgatataaagagtgccctgactcctgctcagctcagcactccaccaaagcctctgcctcagccttactgtgagtctggtaagtgtcggatggtagaaccagggttgggactcgggacctccaacagcatacgatgtggtgggggtgggcagcctgggtgggggtgggcattactctggggctggattcagctggactttcattctagggggactcgagtcagagtactgagagaaaagtgccttggcacagaagtgcagaacagagagtaatcatcctatgtcccatcttttcttgtgaccatatttttggatttgtgtgtgagagagaattatggaagggaggaggggaatagcattcaatttctttcctaaacctcttgggttttgacagaccatcattttgccttctttatggagggagaggttcagggaagagcttccaccttctggctatgctgcacagagggatggcagaatggggaaacctttctatttggagaaacctaggcagagctgggacaggaaaactcaacttagaagtataagacttggaagaacaacctccaactctcagcaaccttccagctcccgcagccccaccccagacacaaggactgcagctaaacctcagaaggtcaggagagaaagcagccctggggttgaataggccaacctgctggctttacaggggggaaaaccgaatcccaggagactaagtgacatgcccagaaacacacagcattccaatgggagattgaggcctagagcatgtcctgtggctccagtctggaggtcacaccatgacctcttagatcctctctggcacggcctataggttttctaggacttggtgttctccaagagacatttcattccctaaggccttactcctcactgtgacataatcccagaacgcatctctgctccttggtcagtgaagcgatgagggtggacacaaggactagacaagagcagacagtgagctggcacctgacccacccttgcagaacagccctgcagacagatctccttgttggctctcacctgggaacaaggaggctcctaggaggacctttctctgcccctccacatttccacccttctctctctgctgcttttgggaaatggtagtccagaggtggtaggacagtaccctgcccaagggaagaggggatgctaaaaaaccagatacttctgcagattcccaaggtttcatctatttcctttgccttcagcctgtgcatcagacctcttctgtctttcaggttgacagtagcttctaagcccggg

SEQ ID NO: 3 - DNA, 658 bp, Homo sapiens
ccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcga

SEQ ID NO: 4 - DNA, 1208 bp, Homo sapiens
  misc_feature (287)..(287): n is a, c, g, or t
ggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacngcctgggttgcaaaggagcacagtgctcatccagatccaaccccctgctatgtgcaggnntcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacaggctgggtcgtaggactgggacaggcagctggagaattgccatgtagatgttcatacaatcgtcaaatcatgaaggctggaaaagnnctccaagatccccaagaccaaccccaacccacccaccgtgccactggccatgtccctcagtgccacatccccacagttcttcatcacctccagggacggtgacncncncctcctccgtggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 5 - DNA, 248 bp, Homo sapiens
atcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagat

SEQ ID NO: 6 - DNA, 496 bp, Homo sapiens
atcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagatatcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagat

SEQ ID NO: 7 - DNA, 285 bp, Homo sapiens
gtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgac

SEQ ID NO: 8 - DNA, 7669 bp, Homo sapiens
gaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctgggcccagccggccagatctgagctcttacgcgtgctagcccgggctcgatcgagatctgcgatctaagtaagcttatattccatgctagggttctggtgttggtgcgtggggttggggtgggactgcagaagtgccttttaagattatgtgattgactgatctgtcattggttccctgccatctttatcttttggattcccctcggaggaggggaggaaggagtttcttttgggttttattgaatcaaatgaaagggaaagtagaggtgttcctatggaggggaggaaggagtttcttttgggttttattgaatcaaatgaaagggaaagtagaggtgttcctatgtcccgggctccggagcttctattcctgggccctgcataagaaggagacatggtggtggtggtggtgggtgggggtggtggggcacagaggaagccgatgctgggctctgcaccccattcccgctcccagatccctctggatatagcaccccctccagtgagcacagcctccccttgccccacagccaacagcaacatgcctcccaacaaagcatctgtccctcagccaaaacccctgttgcctctctctggggaaattgtagggctgggccagggtggggggaccattctctgcagggagattaggagtgtctgtcaggggcgggtggagcggggtggggccctggcttactcacatccttgagagtcctttgctggcagatttggggagcccacagctcagatgtctgtctcagcattgtcttccaagctcctaggccacagtagtggggcgctcccttctctggcttcttctttggtgacagtcaaggtggggttgggggtgacgaagggtcctgcttctcttctaggagcagttgatcccaggaagagcattggagcctccagcaggggctgttggggcctgtctgaggagataggatgcgtcaggcagccccagacacgatcacattcctctcaacatgcctgccggggtctgtggagccgaggggctgatgggagggtggggtgggggccggaagggtttgctttgggaggttgtctgggagattgctgaagttttgatatacacacctccaaagcaggaccaagtggactcctagaaatgtcccctgacccttggggcttcaggagtcagggaccctcgtgtccacctcagccttgcccttgcacagcccagctccactccagcctctactcctccccagaacatctcctgggccagttccacaaggggctcaaacgagggcacctgagctgcccacactagggatgttctgggggtctgagaagatatctggggctggaagaataaaaggcccccctaggcctgttcctggatgcagctccagccactttggggctaagcctgggcaataacaatgccaacgaggcttcttgccatactcggtttacaaaaccctttacatacattgtcgcattggattctcagagctgactgcactaagcagaatagatggtatgactcccactttgcagatgagaacactgaggctcagagaagtgcgaagccctgggtcacagaggcgtaaatgcagagccaggacccacctgaagacccacctgactccaggatgtttcctgcctccatgaggccacctgccctatggtgtggtggatgtgagatcctcaccatagggaggagattagggtctgtgctcagggctggggagaggtgcctggatttctctttgatggggatgttggggtgggaatcacgatacacctgatcagctgggtgtatttcagggatggggcagacttctcagcacagcacggcaggtcaggcctgggagggccccccagacctccttgtctctaatagagggtcatggtgagggaggcctgtctgtgcccaaggtgaccttgccatgccggtgctttccagccgggtatccatcccctgcagcagcaggcttcctctacgtggatgttaaaggcccattcagttcatggagagctagcaggaaactaggtttaaggtgcagaggccctgctctctgtcaccctggctaagcccagtgcgtgggttcctgagggctgggactcccagggtccgatgggaaagtgtagcctgcaggcccacacctccccctgtgaatcacgcctggcgggacaagaaagcccaaaacactccaaacaatgagtttccagtaaaatatgacagacatgatgaggcggatgagaggagggacctgcctgggagttggcgctagcctgtgggtgatgaaagccaaggggaatggaaagtgccagacccgccccctacccatgagtataaagcactcgcatccctttgcaatttacccgagcaccttctcttcactcagccttctgctcgctcgctcacctccctcctctgcaccaagggcgaattccagcacactggcggccgttactagtggatccgagctcgcggccgcgatatcgctagctcgaggagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactggtcgagatcgggagatctggcctccgcgccgggttttggcgccccccgcgggcgccccctcctcacggcgagcgctgccacgtcagacgaagggcgcacgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagaccggaccgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacactcgacctcgaaacttgtttattgcagcttataatggttacaaataaagcaatagcatcacaaatttcacaaataaagcatttttttcactgcattctagttgtggtttgtccaaactcatcaatgtatcttatcatgtctgaattcccggggatcctctagatgcatgctcgagcggccaattcggcttatcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaagatatcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaagataagccgaattggccgccagtgtgatggatatctgcagaattcgcccttgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacaagggcgaattccagcacactggcggccgttactagtggatcaattcggcttatcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaagatatcggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaagataagccgaattgatccgagctcggtaccaagcttcgacctgcagg